(19) United States
(12) Patent Application Publication
(10) Pub. No.: US 2019/0350827 A1
(43) Pub. Date: Nov. 21, 2019

(54) TEETH WHITENER PRE-TREATMENT COMPOSITIONS AND METHODS

(71) Applicant: JASIBO, LLC, Las Vegas, NV (US)

(72) Inventor: Jennifer Jablow, New York, NY (US)

(21) Appl. No.: 16/414,002

(22) Filed: May 16, 2019

Related U.S. Application Data
(60) Provisional application No. 62/672,345, filed on May 16, 2018.
(60) Provisional application No. 62/795,200, filed on Jan. 22, 2019.

(51) Int. Cl. A61K 8/365; A61K 8/27; A61K 8/22; A61Q 11/00; A61K 8/41

(52) U.S. Cl. CPC A61K 8/365; A61K 8/27; A61K 8/22; A61K 2800/81; A61K 8/41; A61K 2800/884; A61Q 11/00

(57) ABSTRACT

Composition and methods that include oral care compositions that are a pre-treatment of the tooth surface to accelerate or catalyze teeth whitener compositions, the teeth whitener pre-treatment composition including a mixture of a metal salt catalyst and a pH adjuster.

### CROSS-REFERENCE TO RELATED APPLICATIONS

This application claims priority to and the benefit of U.S. Provisional Patent Application Ser. No. 62/672,345 filed May 16, 2018 and U.S. Provisional Patent Application Ser. No. 62/795,200 filed Jan. 22, 2019, the disclosures of which are incorporated herein by reference in their entirety.

### FIELD

The aspects of the disclosed embodiments generally relate to dental and oral care products, and in particular compositions and methods to accelerate a teeth whitener composition.

### BACKGROUND

Whiteness of teeth can be an important cosmetic desired element. Common teeth-bleaching method can include oxidizing agents such as hydrogen peroxide, which can be used in a liquid form, and which is mixed with an anhydrous carrier containing glycerin and/or propylene glycol and/or polyethylene glycol. Another oxidizing agent is carbamide peroxide (urea hydrogen peroxide) which can be dissolved in ethanol and which upon contact with water dissociates into urea and hydrogen peroxide. Hydrogen peroxide in the presence of water dissociates into water and an oxygen free radical, the latter being highly reactive and, as a result, react with the stain molecules on teeth making them water soluble and/or transparent or both.

Another approach is to incorporate the oxidizing agent into a strip and have the patient wear the strip intermittently over a period of, for example, two weeks. This approach also has the disadvantage of being awkward for the patients to wear, and many patients wearing the strips also experience tooth sensitivity.

It would be advantageous to have a method of teeth whitening that is more efficient and less time consuming.

### BRIEF DESCRIPTION OF THE DRAWINGS

The accompanying drawings illustrate presently preferred embodiments of the present disclosure, and together with the general description given above and the detailed description given below, serve to explain the principles of the present disclosure.

FIGS. 1A and 1B are illustrations of an illuminating device embodiment of the present disclosure;

FIG. 2 is an illustration of a mouthpiece embodiment;

FIG. 3 is an illustration of illuminator embodiment;

FIG. 4 is an illustration of an illuminating device embodiment of the present disclosure;

FIG. 5 is an illustration of showing the difference in light penetration between the embodiment of FIG. 3 with dual LED/cylindrical lens and an embodiment without cylindrical lens; and

FIGS. 6A and 6B are illustrations of an illuminating device embodiment of the present disclosure.

### SUMMARY

In one embodiment, a teeth whitener pre-treatment composition is provided. The teeth whitener pre-treatment composition includes a metal salt catalyst in an amount of from about 0.05 wt % to about 20 wt % and a pH adjuster in an amount of from about 5 wt % to about 99.5 wt %.

In another embodiment, a teeth whitener pre-treatment composition is provided. The teeth whitener pre-treatment composition includes magnesium gluconate in an amount of from about 0.05 wt % to about 20 wt % and a pH adjuster including sodium bicarbonate in an amount of from about 5 wt % to about 99.5 wt %.

In another embodiment, a method of whitening teeth is provided. The method includes applying a teeth whitener pre-treatment composition to a tooth surface and applying a teeth whitener composition including an oxidizing agent to the tooth surface after the teeth whitener pre-treatment composition is applied. The teeth whitener pre-treatment composition includes a metal salt catalyst in an amount of from about 0.05 wt % to about 20 wt % and a pH adjuster in an amount of from about 5 wt % to about 99.5 wt %

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The term “pharmaceutically acceptable” means that which is useful in preparing a pharmaceutical composition that is generally non-toxic and is not biologically undesirable and includes that which is acceptable for veterinary use and/or human pharmaceutical use.

The term “dentally acceptable” means the compound, substance or device may be administered to or into the oral cavity and/or surfaces of the oral cavity, including the teeth and gums, without substantial harmful effects to the oral cavity and/or its surfaces.

The compositions, ingredients used therein and methods included herein are preferably pharmaceutically acceptable and/or dentally acceptable.

The terms “%”, “% by weight”, “weight %” and “wt %” are all intended to mean unless otherwise stated, percents by weight based upon a total weight of 100% end composition weight. Thus 10% by weight means that the component constitutes 10 wt. parts out of every 100 wt. parts of total composition.

Compositions and methods of the present disclosure include embodiments having oral care compositions that are a pre-treatment of the tooth surface to accelerate or catalyze teeth whitener compositions, the latter that can be used to oxidize stains (e.g. organic stains) on the teeth, for example, those teeth whitener compositions that include a peroxide base tooth whitening agent.

Peroxide and other teeth whitening agents are known to have a whitening effect on the teeth by applying them to a tooth surface in various forms, such as, for example, tray-based gels, peroxide strips and paint on whiteners. By applying a teeth whitener pre-treatment composition including a mixture of a metal salt catalyst and a pH adjuster or a pH adjuster alone to the tooth surface prior to applying the teeth whitener compositions, the operation and effect of the teeth whitener composition can be improved (e.g., accelerated). Such improvement may be attributable to the tooth whitener pre-treatment composition speeding up the breakdown of the teeth whitener compositions (i.e. for a peroxide base teeth whitener composition, the breakdown of peroxide into oxygen radicals to oxidize the stains in the tooth surface). The pH adjuster may raise the pH to a desired level and a metal salt catalyst may catalyze the peroxide breakdown.

The teeth whitener pre-treatment composition can include separate application of the metal salt catalyst and a pH adjuster to the tooth surface or application of a mixture of the metal salt catalyst and a pH adjuster to the tooth surface or a pH adjuster alone to the tooth surface. Where the metal salt catalyst and a pH adjuster are separately applied to the tooth surface, either can be applied first.

The mixture or separate metal salt catalyst and a pH adjuster or pH adjuster alone can be administered, for example, in a powder form, paste form, gel form, a rinse form or spray form. For these forms for administration or application to the teeth that are not in substantially dry powder form, the mixture or separate metal salt catalyst and a pH adjuster or a pH adjuster alone can be also include vehicles, such as, for example, glycerin, propylene glycol, polyvinylpyrrolidone (PVP), distilled water or combinations thereof. If the mixture or separate metal salt catalyst and a pH adjuster or pH adjuster alone are administered in powder form, excipients or other vehicle components are not necessary.

Dentally or pharmaceutically acceptable metal salt catalysts that may be employed include, for example, magnesium gluconate and magnesium oxide zinc oxide, zinc gluconate, iron oxide, ferrous gluconate, ferric chloride (with ferric chloride, not enough to cause an exothermic reaction) or a combination thereof. For the aspects of the disclosed embodiments, magnesium gluconate and magnesium oxide can be employed at concentrations of from about 0.05 weight % to about 20 weight %, preferably from about 0.15 weight % to about 5 weight %, more preferably from about 0.15 weight % to about 1 weight %.

Oral or pharmaceutically acceptable pH adjusters that may be employed include, for example, sodium bicarbonate, sodium hydroxide, potassium hydroxide, triethanolamine (an organic compound composed of a tri-alcohol & an amine), baking soda or a combination thereof. For the aspects of the disclosed embodiments, sodium bicarbonate can be employed at concentrations of from about 5 weight % to about 99.5 weight %, preferably from about 10 weight % to about 80 weight %, except when it is in powder form where it is preferably from about 90 weight % to about 99.5 weight %. For a combination of sodium bicarbonate and sodium hydroxide, sodium bicarbonate can be employed at concentrations preferably from about 15 weight % to about 79 weight % and sodium hydroxide can be employed at concentrations preferably from about 1 to about 5).

The teeth whitener pre-treatment composition should be at a pH of above about 7.5, preferably above a pH of about 8, more preferably above a pH of about 9 with a maximum pH of less than about 11.

The teeth whitener compositions can be applied to the tooth surface after the teeth whitener pre-treatment composition is applied to the tooth surface. The teeth whitener compositions can include peroxide teeth whitener compositions and can be, for example, hydrogen peroxide, carbamide peroxide, calcium peroxide, sodium perborate, sodium percarbonate, urea peroxide or any mixture thereof that is placed on the tooth in the second step. The delivery of the teeth whitener compositions in the second step after the teeth whitener pre-treatment composition and can be applied in gel in tray form, adhesive strips form, gel painted on form or gel placed on a toothbrush and used to brush the teeth to be treated.

The teeth whitener pre-treatment composition can be administered to the teeth for a period of time ranging from about 30 seconds to about 2 minutes, preferably about 1 minute prior to the administration of the teeth whitener composition. When the teeth whitener pre-treatment composition is administered to the teeth prior to the administration of the teeth whitener composition, for a hydrogen peroxide base teeth whitener composition, hydrogen peroxide can be employed at concentrations of from about 3 weight % to about 30 weight %, preferably from about 12 weight % to about 14 weight %. For a carbamide peroxide, calcium peroxide or urea peroxide base teeth whitener composition, each or a mixture can be employed at concentrations of from about 9 weight % to about 48 weight %, preferably from about 36 weight % to about 42 weight %.

Where the teeth whitener pre-treatment composition is administered to the teeth prior to the administration of the teeth whitener composition and the teeth whitener composition includes a hydrogen peroxide base concentration of about 14 weight %, the treatment regimen can range from about 1 ten minute session to about 14 ten min sessions completed over a span of a several days to a week.

One embodiment of the present disclosure is a teeth whitener pre-treatment composition including sodium bicarbonate at a concentration of from about 5 weight % to about 99.5 weight %, preferably from about 40 weight % to about 80 weight %, and magnesium gluconate at a concentration of from about 0.05 weight % to about 20 weight %, preferably from about 0.15 weight % to about 5 weight %, more preferably from about 0.15 weight % to about 1 weight %.

Other embodiments of the present disclosure include a method of applying at least one of the teeth whitener pre-treatment compositions disclosed herein to a tooth, teeth or other oral surfaces. Still other embodiments include a method of applying at least one of the teeth whitener pre-treatment compositions of the present disclosure to a tooth, teeth or other oral surfaces prior to applying a teeth whitener composition, as part of a teeth whitening treatment regimen (a teeth whitening treatment regimen including applying a teeth whitener composition to the tooth, teeth and other oral surfaces). Still other embodiments include a method of applying at least one of the teeth whitener pre-treatment compositions of the present disclosure to a tooth, teeth or other oral surfaces followed by a teeth whitening treatment regimen. Still more embodiments include a method of applying at least one of the teeth whitener pre-treatment compositions of the present disclosure to a tooth, teeth or other oral surfaces followed by applying a teeth whitener composition to the same a tooth, teeth or other oral surfaces to which was applied the at least one of the teeth whitener pre-treatment compositions of the present disclosure.

Other embodiments of the present disclosure include a method of accelerated breakdown of peroxide into oxygen radicals by applying at least one of the teeth whitener pre-treatment compositions of the present disclosure to a tooth, teeth or other oral surfaces followed by applying a teeth whitener composition including peroxide to the same to a tooth, teeth or other oral surfaces. Still other embodiments include a method of raise the pH to a desired level (above about 6.5, preferably above a pH of about 8, more preferably above a pH of about 9 with a maximum pH of less than about 11) and catalyzing the peroxide breakdown of a teeth whitener composition including peroxide by applying at least one of the teeth whitener pre-treatment compositions of the present disclosure to a tooth, teeth or other oral surfaces followed by applying a teeth whitener composition including peroxide. Still other embodiments include a method of priming a tooth or teeth surface for a teeth whitening treatment regimen by applying at least one of the teeth whitener pre-treatment compositions of the present disclosure to a tooth, teeth or other oral surfaces prior to applying a teeth whitener composition.

Optional ingredients include abrasives, colorants, flavoring agents, preservatives, chelating agents, absorbents (bulking agents), binding agents, skin or oral tissue-conditioning agents, skin protectant agents, anti-sensitivity agents and sweetening agents.

Sweetening agents can include, for example, saccharin, dextrose, sucrose, sucralose, lactose, maltose, levulose, aspartame, Stevia, sodium cyclamate, D-tryptophan, dihydrochalcones, acesulfame and mixtures thereof. Sweetening agents can be generally used at levels of from about 0.005 wt % to about 5 wt %, by weight of the composition, preferably from about 2 wt % to about 3 wt %.

Abrasives can include, for example, sodium bicarbonate granular, calcium carbonate, calcium phosphate, bentonite (aka bentonite clay), kaolin clay, hydrated silica and mixtures thereof. Abrasives can be generally used at levels of from about 40 wt % to about 95 wt %, by weight of the composition.

Flavoring agents can include, for example, sucralose, spearmint (e.g. spearmint native oil), peppermint (e.g., peppermint oil) and mixtures thereof. Flavoring agents can be generally used at levels of from about 0.1 wt % to about 2 wt %, by weight of the composition.

Preservatives can include, for example, potassium sorbate, sodium benzoate, grapefruit seed extract and mixtures thereof. Preservatives can be generally used at levels of from about 0.5 wt % to about 2 wt %, by weight of the composition.

Chelating agents can include, for example, disodium EDTA, tetrasodium polyphosphate, hexametaphosphate, pentasodium triphosphate, tetrapotassium polyphosphate, and mixtures thereof. Chelating agents can be generally used at levels of from about 0.1 wt % to about 10 wt %, preferably about 0.1 wt % to about 4.5 wt %, about 0.22 wt %, by weight of the composition.

Absorbents (bulking agents) can include, for example, bentonite, hydrated silica, kaolin clay, and mixtures thereof. Absorbents (bulking agents) can be generally used at levels of from about 1 wt % to about 10 wt %, about 1 wt % to about 4.5 wt %, preferably about 1.45 wt %, by weight of the composition.

Skin or oral tissue-conditioning agents can include, for example, glycerin, hydrated silica, xylitol, xanthan gum, aloe vera and mixtures thereof. Skin-conditioning agents can be generally used at levels of from about 2.5 wt % to about 7 wt %, by weight of the composition.

Anti-sensitivity agents can include, for example, potassium nitrate potassium citrate and arginine and mixtures thereof. Anti-sensitivity agents can be generally used at levels of from about 1 wt % to about 5 wt %, by weight of the composition.

Still other embodiments include a method of applying at least one of the teeth whitener pre-treatment compositions of the present disclosure to a tooth, teeth or other oral surfaces, followed by applying a teeth whitener composition including peroxide to the same tooth, teeth or other oral surfaces and followed by irradiating the same tooth, teeth or other oral surfaces with light, e.g., blue light, indigo light and violet light.

The aspects of the present disclosure are directed to a portable lightsource and a dental bleaching method that overcome certain shortcomings associated with existing whitening products and methods. The lightsource comprises a violet/indigo light illuminator to accelerate teeth bleaching agents. The teeth are separately preconditioned with a bleaching material and, optionally, a catalyst. Specifically, application of the methods and devices of the present disclosure result in an increased whitening rate and shorter home treatment times.

Shorter electromagnetic wavelengths in the violet/indigo bands about 400 nm to about 418 nm (preferably about 405 to about 410 nm) are useful in the process of bleaching teach. It is believed that the emission of violet light (k405-410 nm) coincides with the absorption peak of pigmented molecules in the stains (e.g., yellow stains) that are formed over time. The violet light, thereby, interacting selectively and breaking up the stain molecules into smaller and colorless ones. The speed at which the violet/indigo (preferably violet) light interacts with these stain molecules increases thousands of times, thus accelerating the breakup process. If fragments of the stain molecules do not rearrange, the molecules stop absorbing the violet/indigo (preferably violet) light and the colored center of the stain molecules disappear. This process can occur at higher or lower intensity in many objects with pigmented molecules. Dental pigment molecules (such as stain molecules) are photoreceptive, and, therefore, highly reactive to light. Their molecular chains are long and with sequences of chemical bonds that dislocate electrons and are very susceptible to absorption of shorter wavelengths, such as the violet light.

An advantage of the technique using direct violet/indigo (preferably violet) light is the good aesthetic result obtained after just a few short treatment sessions, preserving the enamel and dental structures. A violet/indigo (preferably violet) photocatalytic bleaching system can also be used in light-activated bleaching procedures using different concentrations of peroxide in bleaching agents, promoting excellent results.

It is believed that violet light has more energy carried in its photons and is more absorbed on the dental surface due to its physical characteristics. These physical violet light characteristics are believed to be responsible for larger surface absorption of light and breaking the pigments (bleaching) that stain teeth. It is also believed that violet light has the characteristic of not having a demineralization effect on the teeth and when used in a fractional way with intervals of time without continuous application does not cause heating of the teeth.

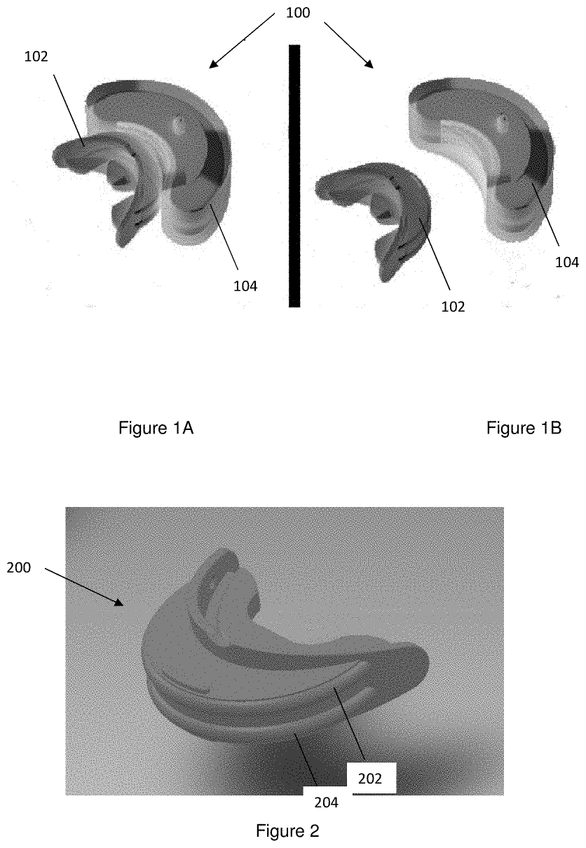

FIGS. 1A and 1B illustrate one embodiment of an illuminating device 100 comprised of a removable mouthpiece 102 that acts as the lightguide for precisely directing the light to the teeth and an illuminator 104, such as, for example an LED lightsource, that generates the light. As used in the present disclosure, the illuminator and LED lightsource can include one or more light generating elements. The optically clear mouthpiece 102 snaps into the illuminator 104 during treatment as in FIG. 1A and is removed as in FIG. 1B for cleaning after each treatment. This design allows for the mouthpiece 102 to be replaced without replacing the other elements of the stem. Each mouthpiece 102 may also be individualized to fit precisely the dental surface. It also allows for use of multiple mouthpieces so that the device may be shared among multiple family members much the same way an electric toothbrush is shared. The detachable lightsource 104 can be powered by a rechargeable battery.

FIG. 2 illustrates one embodiment of the mouthpiece illustrated in FIGS. 1A and 1B in which mounthpiece 200 includes a dual cylindrical lens 202 and 204 that couples the light from a dual row of light generating elements in the lightsouce unit (i.e., an illuminator), including for example, LEDs, precisely onto the teeth. The top and bottom rows of, for example, LEDs from the light source are focused through the upper and lower cylindrical lens 202 and 204 onto the upper and lower row of teeth respectively. The mouthpiece is removable and consumable and is designed to facilitate light activation of a catalyst without transmitting heat to the teeth.

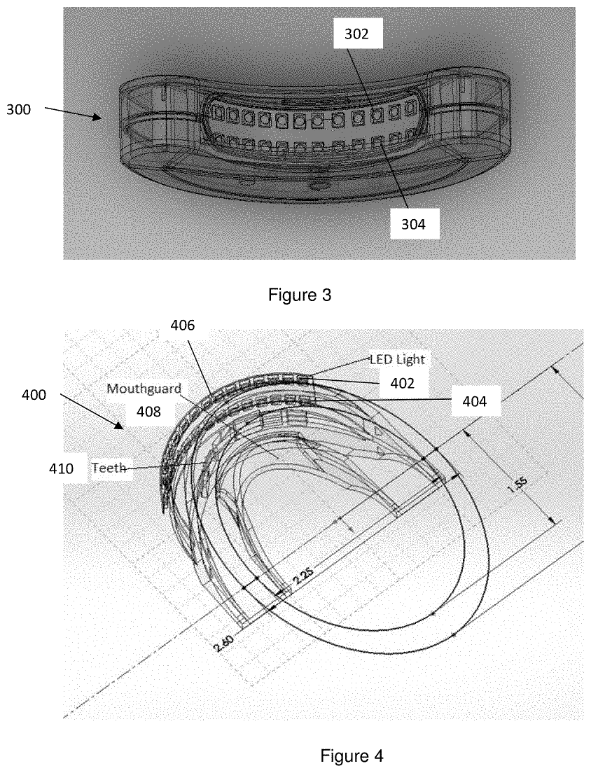

FIG. 3 illustrates one embodiment the illuminator 300 itself which can be used in conjunction with mouthpiece 200 and includes a dual row light generating elements, for example, LEDs, configuration 302 and 304 which may mate with the dual cylindrical lens array 202 and 204 within the mouthpiece 200. The light generating elements 302 and 304 are formed into a curved configuration to mate precisely with the curvature of the lens assemblies 202 and 204 on the front surface of the mouthpiece 200.

FIG. 4 illustrates an embodiment illustrating the novel optical design of the embodiment illustrated in FIGS. 2 and 3 including illuminating device 400, the dual light generating element arrays 402 and 404 of the illuminator 406 and the dual cylindrical lens of mouthpiece 408 which provides optimum irradiance on each row of teeth 410, and minimizes irradiance on the gums and other soft tissues.

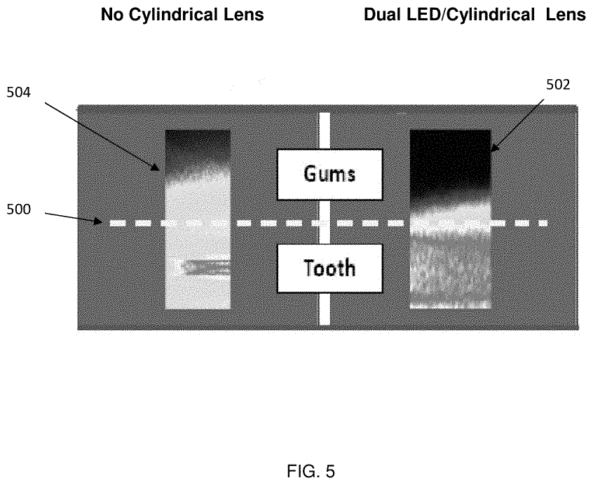

FIG. 5 is an irradiance spectrum demonstrating that the resulting design increases irradiance on the teeth while minimizing irradiance on the gums for the embodiment of FIG. 3 with dual LED/cylindrical lens compared to an embodiment without cylindrical lens. The nature of the single axis cylindrical optics is that it also creates high angle irradiance parallel to the direction of the array, which efficiently irradiates the spaces and cracks between the teeth. Also, there is a greater amount of irradiance penetrating the tooth and less penetrating the gum past the gum line 500 as indicated by the dark area 502 in the dual LED/cylindrical lens data which indicates that no or mimimal light is penetrating into that area of the gum. The colored areas 504 of the no cylindrical lens data indicate that irradiance has penetrated farther past the gum line than where the dual LED/cylindrical lens is used.

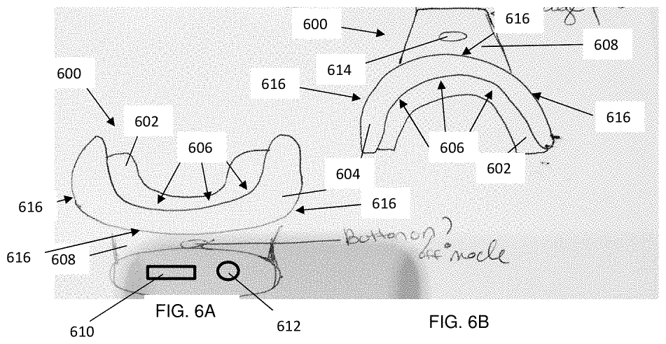

FIGS. 6A and 6B illustrate another embodiment of an illuminating device 600 comprised of a mouthpiece 602 which when positioned for treatment, the teeth bite down on thereby placing the illuminator 604 adjacent the front tooth surface and proximal to the teeth along the inner border 606 of illuminator 604 that includes light generating elements, such as, for example an LED lightsource or plurality of LED light sources, that generates the light. The embodiment of FIGS. 6A and 6B may also include a control/power unit 608 that can include apparatus to power and control the features including the light generating elements as well as other features, such as, for example, apparatus to impose vibration or sonic waves on the teeth being treated and/or apparatus to pulsate, have a continuous strobe or any combination thereof of the light generation elements during the treatment period. The control/power unit may also include one or more charging ports that may be different connector formats, illustrated as connector ports 610 and 612 and on/off button or switch 614. In this embodiment, the lips can fit over the outer boundary 616 of illuminator 604.

Another embodiment of the teeth whitener pre-treatment composition can also optionally include a photocatalytic agent, for example, a zinc compound (pharmaceutically and/or dentally acceptable) such as, for example, zinc oxide and zinc gluconate and can be used when light, including violet light, is part of the whitening process as is disclosed herein. The increased whitening rate and efficiency of the present disclosure also refers to improved photocatalytic activity of one or more photocatalytic agents added to the teeth whitener pre-treatment composition that is applied to the tooth surface before exposure to irradiation. The photocatalytic agent can be present in an amount of ranging from about 0.5 wt % to about 8 wt % by or about 1 wt % to about 5 wt %. In a preferred embodiment the photocatalytic agent is zinc oxide (ZnO). The photocatalytic agent may be added to the teeth whitener pre-treatment composition before or during the pre-treatment. In a preferred embodiment the ZnO is included in the teeth whitener pre-treatment composition that is applied to the tooth surface. The ZnO concentration can be between about 0.5 to about 5% by weight or about 1% by weight. Another embodiment of the disclosure relates to a method of whitening wherein said zinc oxide is a powder USP in a gel or paste formulation of the teeth whitener pre-treatment composition wherein the average zinc oxide particle size is between 0.1 microns to 100 microns, more preferably 0.5 microns to 100 microns, most preferred 10 microns.

The aspects of the present disclosure are also directed to a dental bleaching method using a lighsource that overcomes certain shortcomings associated with existing whitening products and methods, including the methods, devices and compositions disclosed in U.S. Pat. No. 9,492,257 the disclosure of which is hereby incorporated by reference in its entirety. The lightsource comprises a violet/indigo light illuminator to accelerate teeth bleaching agents. The teeth are separately preconditioned with a bleaching material and, optionally, a catalyst. Specifically, application of the methods and devices of the present disclosure result in an increased whitening rate and shorter home treatment times. Also, included are the methods, devices and compositions disclosed in U.S. Pat. No. 9,492,257 including teeth whitening, an oxidizing agent, a photocatalytic agent and a lightsource, the disclosure of which is hereby incorporated by reference in its entirety.

Shorter electromagnetic wavelengths in the violet/indigo bands about 400 nm to about 418 nm (preferably about 405 to about 410 nm) are useful in the process of bleaching teeth. It is believed that the emission of violet light (k405-410 nm) coincides with the absorption peak of pigmented molecules in the stains (e.g., yellow stains) that are formed over time. The violet light, thereby, interacting selectively and breaking up the stain molecules into smaller and colorless ones. The speed at which the violet/indigo (preferably violet) light interacts with these stain molecules increases thousands of times, thus accelerating the breakup process. If fragments of the stain molecules do not rearrange, the molecules stop absorbing the violet/indigo (preferably violet) light and the colored center of the stain molecules disappear. This process can occur at higher or lower intensity in many objects with pigmented molecules. Dental pigment molecules (such as stain molecules) are photoreceptive, and, therefore, highly reactive to light. Their molecular chains are long and with sequences of chemical bonds that dislocate electrons and are very susceptible to absorption of shorter wavelengths, such as the violet light.

An advantage of the technique using direct violet/indigo (preferably violet) light is the good aesthetic result obtained after just a few short treatment sessions, preserving the enamel and dental structures. A violet/indigo (preferably violet) photocatalytic bleaching system can also be used in light-activated bleaching procedures using different concentrations of peroxide in bleaching agents, promoting excellent results.

It is believed that violet light has more energy carried in its photons and is more absorbed on the dental surface due to its physical characteristics. These physical violet light characteristics are believed to be responsible for larger surface absorption of light and breaking the pigments (bleaching) that stain teeth. It is also believed that violet light has the characteristic of not having a demineralization effect on the teeth and when used in a fractional way with intervals of time without continuous application does not cause heating of the teeth.

Another embodiment of the present disclosure may include an illuminating device in which the light generating element (e.g., LED) are positioned inside the mouth while focusing the light on the teeth in order to, for example, bring the light generating elements closer to the teeth. Other embodiments of the illuminating device of the present disclosure may also include apparatus to impose vibration or sonic waves on the teeth being treated. Such vibration or sonic waves can be administered, in one embodiment, for example, after the teeth whitener pre-treatment composition and the teeth whitener composition has been applied and for a period of time ranging from about 15 seconds to about 1 minute, preferably from about 15 seconds to about 30 seconds. The illuminating device then applies light for a period of time ranging from about 6 minutes to about 20 minutes, preferably from about 15 minutes to about 20 minutes. The illuminator of embodiments of the present disclosure can include light generating elements numbering as many as 28 and as little as 6 but preferably 16 to 20, light generating units.

The illuminating devices of the present disclosure may include an illuminator LED lightsource that generates a desired intensity, frequency and wavelength of light that is coupled to a removable or non-removeable mouthpiece that acts as the lightguide for precisely directing the light to the teeth.

The light-emitting diode (LED) array can be housed in a waterproof or water-resistant enclosure which has an optically transmitting faceplate in front of the LED array. The portable lightsource can be completely self-contained and includes the LEDs, the rechargeable battery or batteries, all control and recharging electronics, and a heat management system.

The design uses light energy in the violet portion of the electromagnetic spectrum for photocatalysis of from about 400 nm to about 420 nm (preferably about 405 to about 410 nm). The device can use light generating elements, for example, LED lights, that are continuous, pulsate, have a continuous strobe or any combination thereof during the treatment period.

Such LED lights typically have substrate based on zinc selenide (ZnSe), indium gallium nitride (InGaN), silicon carbide (SiC) and silicon (Si). The LED lights may comprise encapsulated clear or colored molded shells. The shells are designed to boost the light emission from the semiconductor core by acting as a diffusing lens, allowing light to be emitted at a much higher angle of incidence from the light cone than the bare chip is able to emit alone. Light size is designed for maximum tooth illumination. Lights may be of varied size depending on tooth dimensions (molar, incisor, genetically small or large, damaged teeth or gums, etc.). Tooth widths are typically 2-9 mm and thus the LED lights may optionally be of variable size so as to maximally irradiate the tooth surface. The lights are also arrayed along a cylindrical axis in which the emitted light is directed perpendicular to the tooth surface at a distance to maximize the incident intensity. Aspects of the present disclosure may also allow the user to perform mobile hands-free treatments with no connecting cords or secondary elements.

Violet/indigo (preferably violet) illumination of the catalyst conditioned teeth may lead to so-called recombination of the catalytic dissociation products. To limit such recombination, reagents may be added and/or the light may be turned off to spatially control the localized production of ions. As such, the light may be made to strobe (turn on and off repeatedly) to reduce the amount of recombination (the combining of substrate charge centers and/or the photocatalytically created reactive species—both of which reduce activity due to recombination). In a preferred embodiment the flashing rate is 33 Hertz with a 5% duty cycle (5% ON time), however the strobe rate can range from about 1 Hz to about 10 kHz and the duty cycle can range from about 1% to about 75%. These ranges can be adjusted to maximize the whitening rate by increasing the short-term irradiance levels required to drive more instantaneous catalytic events, while at the same time spatially and temporally minimizing recombination events.

Embodiments of the present disclosure may also include a dual cylindrical lens array coupled with a curved dual LED array as illustrated in the embodiment of FIG. 2. This design combination maximizes the activity at and around the front of the tooth surface, including the cracks and spaces between the teeth. This design can provide superior light distribution to the anterior teeth. It can also allow for maximum activity at the tooth surface, with minimal activity on the gums and other soft tissue. The lens material may comprise optically clear polyurethane or any optically clear material that transmits blue light including silicone, PMMA (acrylic), polycarbonate, etc.

The present disclosure also encompasses a heat management system that stores the heat within the lightsource so that it is not conducted or radiated to the teeth. Preferably the heat around the teeth is within 1-2 degrees of ambient oral temperature. Normal tooth temperature is about 35 to about 37° C. (95 to 98.6° F.). The thermal reservoir is sufficient in size that it can store the entire heat generated during a treatment regimen.

Embodiment of the present disclosure also allows the user to perform a rapid whitening operation in a completely remote and hands-free manner, not requiring a sink or to be connected in any way to any type of cable, tether, control device, power storage pack, or power management device.

The increased whitening rate and efficiency of the present disclosure also refers to improved photocatalytic activity of one or more photocatalytic agents added to the oxidizing agent that is applied to the tooth surface before exposure to irradiation. In a preferred embodiment the photocatalytic agent is zinc oxide (ZnO) and the oxidizing agent is hydrogen peroxide (H₂O₂). The photocatalytic agent may be added to the oxidizing agent before or during the treatment. In a preferred embodiment the ZnO is added to the tooth surface first, and the H2O2 is applied over the top of the ZnO film. The ZnO concentration can be between about 0.1 to about 25% by weight. In this manner the catalysis is localized specifically to a thin layer at the surface of the tooth for maximum whitening benefit. This method concentrates the catalytic dissociation of H2O2 precisely at the tooth surface and further ensures that the bulk of the H2O2 remains optically transparent so that the light may penetrate to the surface of the tooth for maximum catalytic activity.

Because these UV and other wavelengths are not desirable for soft tissue exposure, violet/indigo (preferably violet) wavelengths are employed. Also, the catalytic conversion efficiency can be dramatically decreased at longer wavelengths. A variety of absorption shifting agents may be used to enhance the efficacy of these longer wavelengths to violet/indigo (preferably violet) wavelengths. In a preferred embodiment fluorescein can be used because of its existing FDA approval status, but any number or combination of additional absorption shifting elements may be employed.

The catalysis process takes place on the surface of the photocatalytic substrate. In a preferred embodiment this substrate is a small particle of ZnO. In a preferred embodiment these particles are applied in a thin film using a dried atomized powder. However, this film may be applied employed using either hydrophobic, hydrophilic, or amphipathic carriers. The efficiency of a singular conversion is governed by the photon wavelength, and is maximum in the violet/indigo (preferably violet) range. The rate of conversion is governed by the number of photons and the number of substrate sites. Therefore both the irradiance and ZnO concentrations are increased to significantly higher levels than is normally employed. The present disclosure utilizes an irradiance of between 3.5 to about 20 mW/cm2, more preferably between 5 to about 20 mW/cm2, more preferably 10 to about 15 mW/cm2. One embodiment of the disclosure utilizes an irradiance of about 11-12 mW/cm2. Another embodiment utilizes an irradiance of greater than 5 mW/cm2. ZnO substrate concentrations of between 0.1 to 25% by weight are common. More preferably the substrate concentrations are between 4 to about 10%, more preferably about 4 to about 6%, most preferably about 5%. In another embodiment, the substrate concentration is greater than about 10% by weight. In general, the specifications for irradiation and substrate concentration may range from about 0.1 to about 50 mW/cm2 and about 0.1 to about 100% respectively.

In addition to wavelength and irradiance, there are additional rate limiting factors, the first of which is the size of the substrate particle. A particle size that is similar to the wavelength of light to be absorbed can be more optimal. Another embodiment of the disclosure relates to a method of whitening wherein said zinc oxide is a powder USP in a gel formulation wherein the average zinc oxide particle size is between 0.1 microns to 100 microns, more preferably 0.5 microns to 100 microns, most preferred 10 microns. This can help increase absorption efficiency, but also helps spatially separate catalytic dissociation to help minimize recombination.

Another embodiment of the disclosure relates to the use of a whitening or bleaching agent such as carbamide peroxide, hydrogen peroxide, calcium peroxide, zinc peroxide, and sodium perborate but preferably hydrogen peroxide. The oxidizing agent, preferably hydrogen peroxide, may be formulated as a gel, dentifrice, paste, thin film, strip, liquid, spray, or paint applied to the tooth labial surface and creates an admixture with said zinc oxide at the tooth interface. Another embodiment of the disclosure relates to a method of whitening wherein the concentration of hydrogen peroxide ranges from about 0.1 to about 35 percent by weight, more preferably about 10 to about 14 percent by weight. The present disclosure may comprise a variety of different oxidizing agents, peroxide types, and concentrations. The preferred embodiment is about 9% H2O2.

The oxidizing agent may also include the addition of other ionic impurities (singularly or in combination) to boost the generation of active oxidizing agents. These may include sodium chloride, potassium chloride, sodium hydroxide, and a host other such agents known to those skilled in the art, including any mixture thereof.

The present disclosure may also incorporate pre or post treatment of desensitizing agents (singularly or in combination) to minimize the sensitivity commonly experienced. These may include potassium nitrate, potassium citrate, and a host other such agents known to those skilled in the art, including any mixture thereof.

Wax formulations are another specific embodiment of the disclosure. Wax includes synthetic and natural waxes such as paraffins, beeswax, ouricury wax, sugarcane wax, retamo wax, lanolin, petroleum jelly, vegetable waxes (including Bayberry wax, candelilla wax, carnauba wax, Castor wax, Esparto wax, Japan wax, Jojoba oil, Ouricury wax, Rice bran wax or Soy wax). Synthetic fatty acid esters such as cetyl palmitate and myricyl palmitate are additional examples of useful waxes. One specific formulation of water, allyl acrylate copolymer and Hydrogen peroxide is of particular interest.

The present disclosure may also include the use of an activation indicator which can change color upon initiation or completion. This may include methylene blue or other such agents known to those skilled in the art, including any mixture thereof.

The present disclosure may also include post treatment applications including nanoparticle pore sealants, remineralizers, germ fighting compounds, breath fresheners, flavorings and sweeteners, or other such agents known to those skilled in the art, including any mixture thereof.

Another embodiment of the disclosure relates to a method of whitening kit for home use of a whitening agent comprising a composition of zinc oxide, a composition of hydrogen peroxide and a light source.

Another embodiment of the disclosure relates to a method of whitening kit wherein said composition of zinc oxide and said composition of hydrogen peroxide are delivered from a single component with 2 separate chambers that mix upon delivery to the tooth surface. The delivery apparatus for the zinc or peroxide includes syringe, ampoule, or tube. Such apparatus may be opaque so as to prevent damage during storage.

Another embodiment of the disclosure relates to the application of the bleaching formulation wherein the treatment period is from 2-10 minutes, more preferably about 5-7 minutes, more preferably 5 minutes or less. Repeat treatment is also preferred with two to ten sessions being common. More preferably 3-7 sessions (five to seven being most common) are sufficient for achieving desired whiteness. Sessions are typically divided by 24 hrs (six to twelve hours also being very common) but can be longer or shorter depending on individual conditions. Total bleaching time is between 20 to 50 minutes, more preferably 35 minutes total, most preferably 25 minutes (wherein each session is approximately 5 minutes).

Still other embodiments include a method of whitening a tooth or teeth surface using a teeth whitening treatment regimen by applying at least one of the teeth whitener pre-treatment compositions of the present disclosure to a tooth, teeth or other oral surface prior to applying a teeth whitener composition and applying a teeth whitener composition as part of a teeth whitening treatment regimen.

Another embodiment is a kit that includes an embodiment of at least one of the teeth whitener pre-treatment compositions of the present disclosure and may also include instructions for use. Another embodiment is a kit that includes at least one of the teeth whitener pre-treatment compositions of the present disclosure and a teeth whitener composition and may also include instructions for use. Another embodiment is a kit that includes at least one of the teeth whitener pre-treatment compositions of the present disclosure, a teeth whitener composition, a light for irradiating the same tooth, teeth or other oral surfaces and may also include instructions for use.

### Example 1—Teeth Whitener Pre-Treatment (Pre-Paste)

Ingredients: sodium bicarbonate granular (abrasive; skin protectant) 48.5 wt %; glycerin (skin-conditioning agent) 32.97 wt %; DI water (skin-conditioning agent) 9 wt %; hydrated silica (skin-conditioning agent) 3 wt %; xylitol (skin-conditioning agent) 1.5 wt %; bentonite (absorbent; bulking agent) 1.45 wt %; sucralose (flavoring agent) 0.85 wt %; potassium sorbate (preservative) 0.62 wt %; sodium benzoate (preservative) 0.6 wt %; xanthan gum (skin-conditioning agent) 0.59 wt %; PVP K-30 (binder) 0.35 wt %; disodium EDTA (chelating agent) 0.22 wt %; magnesium gluconate (skin-conditioning agent) 0.2 wt %; and spearmint native oil (flavoring agent) 0.15 wt %. Each of the ingredients may have additional functions along with the stated functions.

### Example 2—Teeth Whitener Pre-Treatment with Zinc for Use with Light as Part of the Whitening Process

Ingredients: sodium bicarbonate granular (abrasive; skin protectant) 48.5 wt %; glycerin (skin-conditioning agent) 31.97 wt %; zinc oxide (photocatalytic agent) 1 wt %; DI water (skin-conditioning agent) 9 wt %; hydrated silica (skin-conditioning agent) 3 wt %; xylitol (skin-conditioning agent) 1.5 wt %; bentonite (absorbent; bulking agent) 1.45 wt %; sucralose (flavoring agent) 0.85 wt %; potassium sorbate (preservative) 0.62 wt %; sodium benzoate (preservative) 0.6 wt %; xanthan gum (skin-conditioning agent) 0.59 wt %; PVP K-30 (binder) 0.35 wt %; disodium EDTA (chelating agent) 0.22 wt %; magnesium gluconate (skin-conditioning agent) 0.2 wt %; and spearmint native oil (flavoring agent) 0.15 wt %. Each of the ingredients may have additional functions along with the stated functions.

### Example 3—Comparison of Extrinsically Stain Bovine Enamel Treated with Teeth Whitener Composition and with and without Teeth Whitener Pre-Treatment Composition

Teeth whitener pre-treatment composition powder exemplified in Example 1

Teeth whitener composition (Propylene Glycol, Purified Water, Glycerin, Hydrogen Peroxide, Triethanolamine, Carbomer, Xylitol, Flavor, Potassium Nitrate)

The purpose was to evaluate the whitening effect on extrinsically stained bovine enamel of a sponsored supplied combo brush-on pre-treatment composition powder and teeth whitener composition whitening pen vs. the whitening pen alone. Approximately 4-mm squares of dental enamel were cut, using a diamond cutting disk, from bovine permanent incisors. Using a mold, the enamel squares are embedded in clear orthodontic repair resin¹ to provide 1.5 cm square blocks with the labial surface exposed. Test Group treatment procedure: 1) Brush tooth surface with toothbrush dipped in powder for 1 minute; 2) Did not rinse but blotted dry;

3) Paint thin layer of whitening gel on dry tooth surface and leave on for 10 min; 4) Wipe off whitening gel, but did not rinse for 20 min afterwards; 5) Store treated tooth specimens in artificial saliva until next treatment cycle; 6) Repeat Steps 1-4 for each treatment cycle; and 7) Perform 7 treatment cycles with spectrophotometer readings after 3, 4, 5, 6, and 7 days. Control Group treatment procedure: 1) Paint thin layer of whitening gel on dry tooth surface and leave on for 10 min; 2) Wipe off whitening gel, but did not rinse for 20 min afterwards;

3) Store treated tooth specimens in artificial saliva until next treatment cycle; 4) Repeat Steps 1-3 for each treatment cycle; and 5) Perform 7 treatment cycles with spectrophotometer readings after 3, 4, 5, 6, and 7 days.

The difference between the pre-test and post-test readings for each color factor (ΔL*, Δa*, and Δb*) represents the ability of the whitening devices to remove intrinsic tooth stain by whitening. The overall change in stain is calculated using the CIELAB equation ΔE=[(ΔL*)²+(Δa*)²+(Δb*)²]. Each component of the L*a*b* scale also is analyzed separately to determine the specific changes in white-black, red-green, and yellow-blue, respectively.

| | | Baseline | | |
|---|---|---|---|---|
| ID | Gp | L* | a* | b* |
| 1. Powdr/Gel | 1 | 33.25 | 12.40 | 14.93 |
| 2. Gel Only | 2 | 33.56 | 11.82 | 14.26 |

| | 3 treatment | | | 3 treatment difference | | | |
|---|---|---|---|---|---|---|---|
| ID | L* | a* | b* | L* | a* | b* | E |
| 1. Powdr/Gel | 43.20 | 11.76 | 27.04 | 9.95 | −0.63 | 12.11 | 15.81 |
| 2. Gel Only | 37.26 | 12.36 | 19.58 | 3.70 | 0.53 | 5.32 | 6.53 |

| | 4 treatment | | | 4 treatment difference | | | |
|---|---|---|---|---|---|---|---|
| ID | L* | a* | b* | L* | a* | b* | E |
| 1. Powdr/Gel | 45.76 | 10.83 | 27.19 | 12.51 | −1.56 | 12.25 | 17.84 |
| 2. Gel Only | 38.79 | 12.55 | 21.61 | 5.23 | 0.73 | 7.35 | 9.11 |

| | 5 treatment | | | 5 treatment difference | | | |
|---|---|---|---|---|---|---|---|
| ID | L* | a* | b* | L* | a* | b* | E |
| 1. Powdr/Gel | 48.59 | 9.82 | 27.45 | 15.34 | −2.57 | 12.52 | 20.33 |
| 2. Gel Only | 40.25 | 12.27 | 22.77 | 6.69 | 0.44 | 8.51 | 10.90 |

| | 6 treatment | | | 6 treatment difference | | | |
|---|---|---|---|---|---|---|---|
| ID | L* | a* | b* | L* | a* | b* | E |
| 1. Powdr/Gel | 51.34 | 8.78 | 26.20 | 18.09 | −3.62 | 11.26 | 22.10 |
| 2. Gel Only | 41.66 | 12.16 | 24.75 | 8.10 | 0.34 | 10.49 | 13.30 |

| | 7 treatment | | | 7 treatment difference | | | |
|---|---|---|---|---|---|---|---|
| ID | L* | a* | b* | L* | a* | b* | E |
| 1. Powdr/Gel | 53.03 | 8.06 | 25.00 | 19.78 | −4.33 | 10.07 | 23.24 |
| 2. Gel Only | 42.87 | 11.90 | 25.74 | 9.30 | 0.08 | 11.48 | 14.88 |

The following examples demonstrate methods or uses to successfully whiten a patient's teeth using light as included in the present disclosure.

### Example 2

1. Zinc oxide is painted on teeth labial surface in a gel carrier form with the zinc oxide (USP grade) in a concentration of about 4 to about 6%, preferred about 1%.

2. Hydrogen peroxide gel in a concentration of about 9% (can be used at about 12% or about 14%) is painted on the labial surface of the teeth creating an admixture of the zinc oxide and peroxide at the tooth surface (steps 1 and 2 are interchangeable).

3. An illuminator LED lightsource array coupled with a dual cylindrical lens mouthguard pulsating at a frequency of about 33 Hertz with a 5% duty cycle (5% ON time) and a wavelength of about 405 to 410 nm is directed at the tooth surface and away from the oral tissues. The LED lightsource may or may not be a pulsating light.

4. After treatment period (e.g., 5, 7, 30 or 35 minutes) the light source is removed. This procedure may be repeated 3-7 consecutive times (such as daily or twice daily) with light source removed after each treatment (e.g., 5, 7, 30 or 35 minutes). There may be 2 or more treatments conducted.

### Example 3

The zinc and peroxide components are maintained in two separate chambers of a pen or syringe and mix at the delivery tip as the components are applied to the tooth. Once the zinc and peroxide components have been applied to the teeth an LED violet/indigo (preferably violet) light source is directed at the tooth surface and away from the oral tissues.

### Example 4

The zinc and peroxide components are maintained in two separate chambers of a pen or syringe or two separate pens or syringes. Zinc oxide (USP grade) is painted on teeth labial surface in a gel carrier form at a concentration of about 1% to about 6% with the size of the particle ranging from about 0.5 to 10 microns. Hydrogen peroxide gel in a concentration of about 9% to about 14% is painted on the labial surface of the teeth on top of the zinc oxide creating an admixture of the zinc oxide and peroxide at the tooth surface. The teeth are then illuminated as described above in Example 1.

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Thus, while there have been shown, described and pointed out, fundamental novel features of the present disclosure as applied to the exemplary embodiments thereof, it will be understood that various omissions and substitutions and changes in the form and details of devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

1. A teeth whitener pre-treatment composition, comprising:
a. a metal salt catalyst in an amount of from about 0.05 wt % to about 20 wt %; and
b. a pH adjuster in an amount of from about 5 wt % to about 99.5 wt %.

2. The teeth whitener pre-treatment composition according to claim 1, wherein the metal salt catalyst is magnesium gluconate, magnesium oxide or a combination thereof.

3. The teeth whitener pre-treatment composition according to claim 1, wherein the pH adjuster is sodium bicarbonate, sodium hydroxide, triethanolamine (an organic compound composed of a tri-alcohol & an amine) or a combination thereof.

4. The teeth whitener pre-treatment composition according to claim 1, wherein the metal salt catalyst is magnesium gluconate and the pH adjuster is sodium bicarbonate.

5. The teeth whitener pre-treatment composition according to claim 1, wherein the metal salt catalyst is magnesium gluconate is in an amount of about 0.2 wt % and the pH adjuster is sodium bicarbonate is in an amount of about 48.5 wt %.

6. The teeth whitener pre-treatment composition according to claim 1, wherein the composition further includes a photocatalytic agent.

7. The teeth whitener pre-treatment composition according to claim 1, wherein the photocatalytic agent is a zinc compound.

8. A teeth whitener pre-treatment composition, comprising:
a. a metal salt catalyst including magnesium gluconate in an amount of from about 0.05 wt % to about 20 wt %; and
b. a pH adjuster including sodium bicarbonate in an amount of from about 5 wt % to about 99.5 wt %.

9. The teeth whitener pre-treatment composition according to claim 8, wherein the composition further includes a zinc-containing photocatalytic agent.

10. The teeth whitener pre-treatment composition according to claim 9, wherein the magnesium gluconate is in an amount of about 0.2 wt % and the sodium bicarbonate is in an amount of about 48.5 wt %.

11. A method of whitening teeth, comprising:
a. applying a teeth whitener pre-treatment composition to a tooth surface, the teeth whitener pre-treatment composition comprising:
i. a metal salt catalyst in an amount of from about 0.05 wt % to about 20 wt %; and
ii. a pH adjuster in an amount of from about 5 wt % to about 99.5 wt %;
b. applying a teeth whitener composition including an oxidizing agent to the tooth surface after the teeth whitener pre-treatment composition is applied.

12. The method according to claim 11, wherein the metal salt catalyst is magnesium gluconate and the pH adjuster is sodium bicarbonate.

13. The method according to claim 11, wherein the metal salt catalyst is magnesium gluconate is in an amount of about 0.2 wt % and the pH adjuster is sodium bicarbonate is in an amount of about 48.5 wt %.

14. The method according to claim 11, wherein the teeth whitener composition includes hydrogen peroxide in an amount ranging from about 3 wt % to about 30 wt %.

15. The method according to claim 11, wherein at least one of the teeth whitener pre-treatment composition and the teeth whitener composition includes a photocatalytic agent and further includes the step of irradiating irradiating the tooth with light from a lightsource.

16. The method according to claim 15, wherein the photocatalytic agent is a zinc compound.

17. The method according to claim 15, wherein the step of irradiating irradiating the tooth with light from a lightsource takes place after the step of applying the teeth whitener pre-treatment composition.

18. The method according to claim 15, wherein the wavelength of the light from the lightsource ranges from about about 400 nm to about 418 nm.

19. The method according to claim 15, wherein the photocatalytic agent is zinc oxide.

20. The method according to claim 15, wherein the photocatalytic agent includes zinc oxide in an amount ranging from about 0.15 wt % to about 5 wt %.
